(19) United States
(12) Patent Application Publication
(10) Pub. No.: US 2024/0000674 A1
(43) Pub. Date: Jan. 4, 2024

(54) dual-compartment pouch packaging system for a bentonite clay face mask product with a frangible division separating a liquid material and a dry powder material in two individual but connected compartments.

(71) Applicants: Thomas Siewhon Yang, Seattle, WA (US); Dan Uhm, Seattle, WA (US)

(72) Inventors: Thomas Siewhon Yang, Seattle, WA (US); Dan Uhm, Seattle, WA (US)

(21) Appl. No.: 17/726,951

(22) Filed: Apr. 22, 2022

Related U.S. Application Data
(60) Provisional application No. 63/179,787, filed on Apr. 26, 2021.

(51) Int. Cl. A61K 8/19; A61K 8/02

(52) U.S. Cl. CPC A61K 8/19; A61K 8/0212; A61K 2800/882; A61K 2800/87

(57) ABSTRACT

The present invention provides for a unique packaging solution that allows a user to easily mix and prepare a fresh hydrated bentonite clay face mask paste without contamination, special mixing containers, or special mixing utensils. This is accomplished with a dual-compartment pouch packaging system for a bentonite clay face mask product with a frangible division separating a liquid material and a dry powder material in two individual but connected compartments. One benefit and advantage of this method and packaging system is that the dry clay powder is stored in a clean and sterile compartment prior to immediate mixing with a sterile liquid, which is also stored in its own clean and sterile compartment within the individual dual-compartment pouch system. This allows for a preservative and chemical free product.

### SUMMARY OF THE INVENTION

The present invention provides for a unique packaging solution that allows a user to easily mix and prepare a fresh hydrated bentonite clay face mask paste without contamination, special mixing containers, or special mixing utensils. This is accomplished with a dual-compartment pouch packaging system for a bentonite clay face mask product with a frangible division separating a liquid material and a dry powder material in two individual but connected compartments. One benefit and advantage of this method and packaging system is that the dry clay powder is stored in a clean and sterile compartment prior to immediate mixing with a sterile liquid, which is also stored in its own clean and sterile compartment within the individual dual-compartment pouch system.

### DESCRIPTION

### Background of the Invention

### 1. Field of the Invention

The present invention relates to a unique packaging solution to overcome the problems and issues associated with typical preparation and packaging of bentonite clay face mask products, including bentonite clay ready to use products.

### 2. Description of Related Art

Known multi-compartment packing compartments that are similar to this packaging application for other liquid and solid products has not been used to package a sterile liquid and a sterile dry bentonite clay powder for easily mixing and preparing a hydrated bentonite clay paste. For example, see U.S. Pat. No. US20060093765A1 and U.S. Pat. No. 4,798,288.

This packaging solution allows for the end user to easily mix freshly prepared bentonite clay mask paste with less mess and readily use the product which avoids the use of preservatives and introducing cations into the final mixed product. This packaging solution also reduces the possibility of contamination of the liquid component or the dry clay powder component due to improper packaging, storage, or handling during use since both components do not come in contact with the surrounding environment or organic matter including bacteria that may be introduced by contaminated utensils, fingers, or by other means. Other existing “ready to use” hydrated bentonite clay products must include preservatives, and often other chemicals, to prevent (with preservatives) growth of bacteria, fungus, mold, and other pathogens that can be harmful to human health or even deadly if the product were to become contaminated by e-coli bacteria for example.

Bentonite clay cosmetic products have been used as masks for hundreds of years and are known for their ability to detoxify skin and deep cleanse skin pores. Current products consist of jars of dry clay powder that the end user must add a liquid (water, apple cider vinegar, etc.) and with some difficulty mix into a smooth paste before using. Other hydrated products currently contain additional preservative ingredients necessary to avoid bacterial growth in the clay as well as emulsifiers and other unnatural ingredients. As clay is a substrate for bacteria this is an important obstacle to avoid for the safety of the end user. Another issue is that when a hydrated clay product comes in contact with metal it cancels the negative charge of the clay which is thought to be part of what makes the clay effective in detoxification and cleansing of skin pores by adsorption. All of these issues are important in the safety and effectiveness of the mixed clay cleansing and detoxification product. Another issue is having a product that is natural and organic without the addition of other chemicals and ingredients.

### DESCRIPTION OF THE INNOVATION

We are proposing a packaging system which allows for two primary components for a hydrated bentonite clay product, which is a liquid and dry bentonite clay powder. The two separate components are packaged separately and sterilely and then mixed by the end user immediately before use. This also would avoid issues with using metallic filling devices in the hydrated clay product which decreases the effectiveness of the clay.

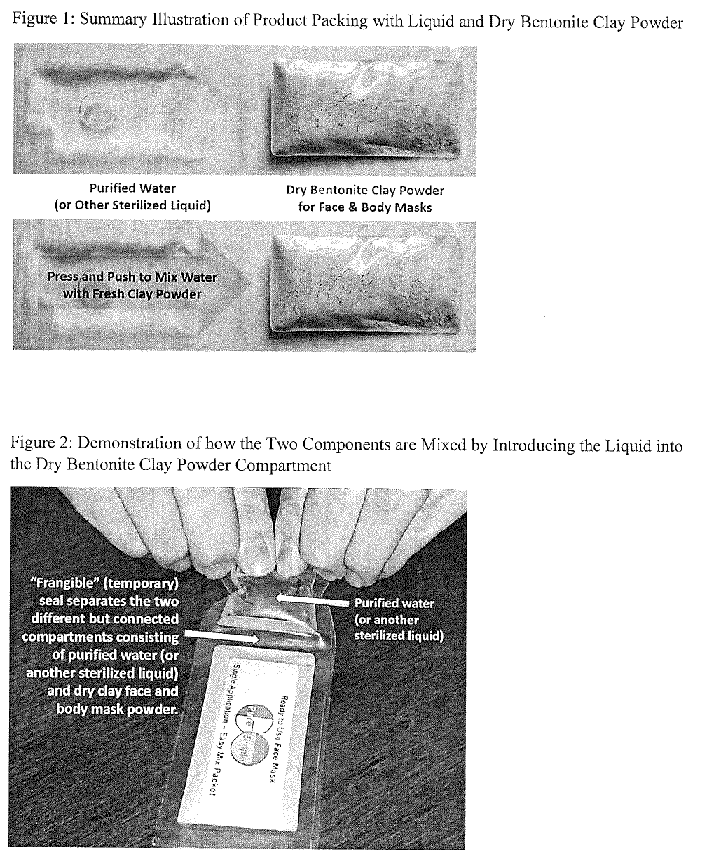

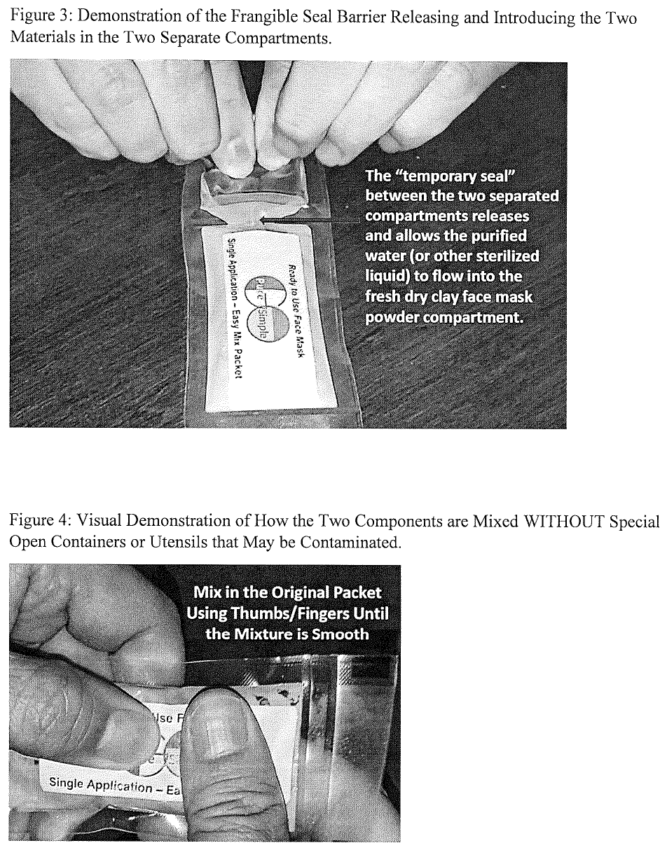

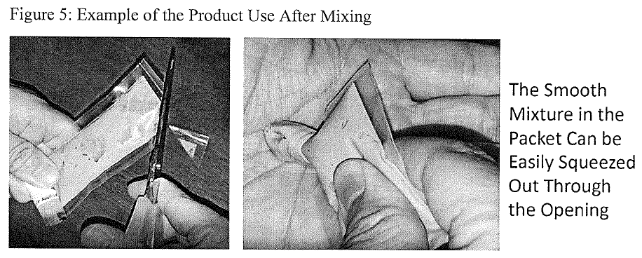

Plastic pouch multi-compartment systems with frangible inner barriers between the compartments have been used for certain other materials and applications including the most common which is typically for two-part liquid products. However, this packaging solution has not been used to package a sterile liquid and a sterile and dry bentonite clay powder for easily mixing and preparing a hydrated bentonite clay paste. A detailed and visual illustration of the innovation can be seen in FIG. 1 through FIG. 5.

1. Dry clay powder is stored in a clean and sterile compartment prior to immediate mixing with a sterile liquid, which is also stored in its own clean and sterile compartment within the individual dual-compartment pouch system.

2. Easily mix freshly prepared bentonite clay mask paste with less mess and readily use the product which avoids the use of preservatives or added chemicals and introducing cations into the final mixed product.

3. This packaging solution also reduces the possibility of contamination of the liquid component or the dry clay powder component due to improper packaging, storage, or handling during use since both components do not come in contact with the surrounding environment or organic matter including bacteria that may be introduced by contaminated utensils, fingers, or by other means.

4. To prevent (without preservatives) growth of bacteria, fungus, mold, and other pathogens that can be harmful to human health or even deadly if the product were to become contaminated by E-coli bacteria for example.

5. Other hydrated products currently contain additional preservative ingredients necessary to avoid bacterial growth in the clay as well as emulsifiers and other unnatural ingredients.

6. When a hydrated clay product comes in contact with metal, it cancels the negative charge of the clay which is thought to be part of what makes the clay effective in detoxification and cleansing of skin pores by adsorption.

7. Two separate components are packaged separately and sterilely and then mixed by the end user immediately before use, which avoids issues with using metallic filling devices in the hydrated clay product which decreases the effectiveness of the clay.

8. Plastic pouch multi-compartment systems with frangible inner barriers between the compartments have been used for certain other materials and applications including the most common which is typically for two-part liquid products.

9. This dual-compartment packaging solution has not been used to package a sterile liquid and a sterile and dry bentonite clay powder for easily mixing and preparing a hydrated bentonite clay paste.
